(19) United States
(12) Patent Application Publication
(10) Pub. No.: US 2023/0241761 A1
(43) Pub. Date: Aug. 3, 2023

(54) A JOINT LOCKING MECHANISM

(71) Applicant: Imperial College Innovations Limited, London (GB)

(72) Inventors: Shen Treratanakulchai, London (GB); Guang-Zhong Yang, London (GB)

(21) Appl. No.: 18/007,207

(22) PCT Filed: Jul. 13, 2021
(86) PCT No.: PCT/GB2021/051784

§ 371 Date: Jan. 27, 2023

(30) Foreign Application Priority Data
Jul. 28, 2020 (GB) 2011682.8

(51) Int. Cl. B25J 3/04; A61B 34/35; B25J 13/02

(52) U.S. Cl. CPC B25J 3/04; A61B 34/35; B25J 13/02; A61B 2034/301

(57) ABSTRACT

A joint locking mechanism comprising: a locking gear rotatable about a rotation axis; a locking latch removably engageable with the locking gear and movable relative to the rotation axis between first and second latch positions; and a bias couplable to the locking latch such that the locking latch is biased towards the first latch position, wherein in the first latch position the locking latch is engaged with the locking gear such that the locking gear is locked in position relative to the rotation axis and in the second latch position the locking latch is spaced apart from the locking gear such that the locking gear is rotatable about the rotation axis.

This invention relates to a joint locking mechanism for preventing a rotatable joint from rotating, and more particularly although not exclusively, to a joint locking mechanism forming part of a master controller wherein the joint locking mechanism may lock a rotatable joint of the master controller.

A teleoperation system is a remote-control system between a master controller situated locally to the operator and a slave device situated remotely to the operator wherein the teleoperation system allows the slave device to be manipulated by an operator at a distance. Such teleoperation systems may be useful if there is a barrier to direct manipulation. For example, a slave device may be used to carry out a minimally invasive surgical procedure in which direct manipulation of surgical tools by a surgeon is not possible due to restricted access to the patient's internal tissues and organs. Other examples may include military applications such as bomb disposal, emergency service applications such as search and rescue, and scientific research activities such as carrying out tasks in inhospitable environments (e.g. vacuums or high levels of reactivity) where direct manipulation could be hazardous or impossible.

An operator of a teleoperation system may manipulate the master controller and a motion command will be transmitted to the slave device such that the slave robot replicates the operator's local movement at the remote site. As well as control data being transmitted from the master to the slave, feedback data may be transmitted from the slave to the master in the form of video footage, sound recordings and/or sensed data from the remote environment. The feedback data may improve the operator's ability to provide accurate commands in much the same way as an operator would use their natural senses of sight, hearing and touch to carry out actions directly (without a teleoperation system).

However, differences between the kinematics of the master controller and slave device can lead the operator to get lost or awkwardly positioned due to decoupling from the physical world while controlling the slave device. This may be because the master controller and slave device operate on different scales with different ranges of movement relative to one another. For example, a slave surgical device may operate within a workspace just a few centimetres in diameter while the operator may be using the full range of motion that his or her arms allow. Despite the slave surgical device being much smaller in scale it may be capable of a larger range of movement (relative to its scale) than the operator can achieve with the master controller. In other words, the operator's range of movement may represent only a portion of the slave device's range of movement.

To overcome this issue, known teleoperation systems feature a clutch which allows the operator to disengage the master controller from transmitting motion commands to the slave device. This allows the operator to re-couple with the physical world and reset the master controller to a comfortable position before re-engaging the master controller so that it resumes transmitting motion commands to the slave device. The operator may therefore return to a position that is comfortable without the slave device moving, the operator may then be able to access a different portion of the slave device's workspace.

However, an issue with such teleoperation systems is the resulting misalignment between the master controller and the slave device. The decoupling of translational position between the master controller and slave device may have minimal effect on the operator perception of the slave device. Whereas, misalignment between the orientation of the master controller and the slave device may be more difficult to perceive. For example, the operator may lose perception that the slave device was oriented downwardly before clutching and after clutching may assume that a forward movement of the master controller will result in a forward movement of the slave device when it may in fact continue in the downwards direction set prior to clutching.

In order to avoid misalignment of orientation between the master controller and slave device, known master controllers comprise ‘active’ joints wherein the stiffness of each active rotatable joint is actively varied depending on forces that are applied to it. When no forces are applied to the master controller by an operator, the master controller sets the stiffness of the active joints such that each joint holds its current rotational position despite environmental forces such as gravity acting on it. In other words, an operator of the active master controller could let go of the handles and they would remain in the same position and orientation. Conversely, when the master controller senses a force being applied to an active joint by a user, the stiffness of that joint is reduced so that the user can cause it to rotate freely.

When such master controllers are disengaged from controlling the slave device, via clutching, the joints of the master controller that correspond to orientation of the slave device may be locked in position by increasing the stiffness of those joints such that the operator is unable to cause rotation of them. The orientation of the master controller may therefore be maintained at the orientation pre-clutching such that the master controller remains aligned with the slave device throughout the clutching process.

However, motorised components required to provide the active variation of joint stiffness in known active joints can result in the joints becoming heavy and bulky. This may be disadvantageous as master controllers are to be operated by a human user and therefore should ideally be small and light so that the user can manipulate the master controller as easily and naturally as possible.

Master controllers may alternatively comprise ‘passive’ joints wherein the stiffness of each joint is constant, irrespective of different forces being applied to it. Passive joints do not require motorised components and therefore avoid issues of added size and weight. On the other hand, passive joints also lack any inherent means of preventing rotation during a clutching process.

According to a first aspect of the invention there is provided a joint locking mechanism comprising: a locking gear rotatable about a rotation axis; a locking latch removably engageable with the locking gear and movable relative to the rotation axis between a first latch position and a second latch position; and a bias couplable to the locking latch such that the locking latch is biased towards the first latch position, wherein in the first latch position the locking latch is engaged with the locking gear such that the locking gear is locked in position relative to the rotation axis and in the second latch position the locking latch is spaced apart from the locking gear such that the locking gear is rotatable about the rotation axis.

By means of the invention an operator of the joint locking mechanism may selectively prevent or allow rotation of the locking gear about the rotation axis by positioning the locking latch in the first latch position or second latch position respectively. The joint locking mechanism may be applied to a passive joint of a master controller to selectively prevent rotation of the joint, for example during a clutching process to maintain orientation alignment with a slave device.

Further, the locking latch is biased towards the first latch position, in which the locking gear is prevented from rotating about the rotation axis, such that the joint locking mechanism defaults to preventing rotation of the locking gear. The operator is therefore required to actively move the locking latch to the second latch position in order to allow rotation of the locking gear. If the joint locking mechanism is applied to a passive joint of a master controller, the joint may default to a locked position. The master controller may therefore exhibit similar advantages to known master controllers with active joints wherein the joints may hold their current position until an operator intends to manipulate the master controller and releases the joint locking mechanism in order to do so. However, the need for bulky motorised components required of known active joints may simultaneously be avoided.

The locking latch may be engageable with the locking gear by any suitable means. For example, in some embodiments of the invention the locking gear may comprise a plurality of teeth extending from its circumference and the locking latch may comprise a tooth receiving portion configured to receive a tooth of the locking gear when the locking latch is positioned in the first latch position. In other embodiments of the invention the locking latch may comprise a tip receivable within a gap between two teeth extending from the locking gear.

The locking latch may also be movable by any suitable means. For example, in some embodiments of the invention the locking latch may be tendon driven. In order to move the locking latch from the first latch position to the second latch position, a tendon attached to the locking latch may be actuated (by increasing its tension) to pull the locking latch against the bias. Then, in order to move the locking latch back to the first latch position, the tendon may be released so that the bias overrides the tension of the tendon and moves the locking latch towards the first latch position.

The bias may be any suitable means for biasing the locking latch towards the first position, such as a spring, a magnet or a pressurised piston for example.

In other embodiments of the invention the locking latch may be motor driven, electromagnetically driven or pneumatically driven, for example.

In embodiments of the invention the locking latch may be movable between the first latch position and the second latch position in a direction normal to the rotation axis.

In such embodiments of the invention, the locking latch may be movable radially to the locking gear. Therefore, when the locking latch moves to the first latch position while the locking gear is rotating, there may be reduced likelihood of the locking gear slipping past the locking latch and the engagement failing.

In embodiments of the invention the joint locking mechanism may further comprise a latch receptacle fixedly positionable relative to the rotation axis wherein the locking latch is movably receivable within the latch receptacle and the bias is couplable to the latch receptacle such that it extends between the latch receptacle and the locking latch.

In such embodiments of the invention the latch receptacle may comprise one or more internal walls adapted such that, when the locking latch is received within the latch receptacle, the locking latch is confined to movement between the first and second latch positions by the latch receptacle. When the locking latch is in the first latch position, engaged with the locking gear, the internal walls of the latch receptacle may support the locking latch against lateral forces that may be transmitted to the locking latch by the locking gear.

The bias may be a spring (or any other suitable biasing means) couplable to the locking latch and latch receptacle in any way suitable for biasing the locking latch towards the first latch position. For example, in some embodiments of the invention the spring may be attachable between the locking latch and a first end of the latch receptacle wherein the spring is biased towards contracting such that the locking latch is pulled to the first latch position. In other embodiments of the invention the spring may be attachable to the locking latch and a second end of the latch receptacle wherein the spring is biased towards expanding such that the locking latch is pushed to the first latch position.

In embodiments of the invention the locking latch may comprise a plurality of rollers, each roller being rotatable relative to the locking latch and engageable with the latch receptacle.

In such embodiments of the invention each rotatable roller may be engageable with an internal wall of the latch receptacle wherein the rotatable roller may roll clockwise or anticlockwise along the internal wall like a wheel along a road. Accordingly, the plurality of rollers may facilitate movement of the locking latch between the first and second latch positions by rolling along the respective internal walls of the latch receptacle.

In embodiments of the invention the latch receptacle may extend linearly along a latch axis.

In such embodiments of the invention, the locking latch may be movable linearly along the latch axis between the first latch position (proximal to the rotation axis) and the second latch position (distal to the rotation axis).

In embodiments of the invention the latch axis may extend normally to the rotation axis.

In such embodiments of the invention, the latch receptacle extends normally to the rotation axis and may extend substantially parallel to a portion of the master controller arm with which the latch receptacle is associated and which is rotatable about the rotation axis. By virtue of this, the respective portion of the master controller arm may be more sleekly designed as the latch receptacle may extend through the arm, substantially or entirely hidden within the arm, rather than extending across the arm and potentially adding a degree of bulkiness to the design.

In embodiments of the invention the latch receptacle may comprise: a plurality of guide rails; the locking latch may comprise a plurality of guide apertures, each guide aperture slidably engageable with a respective one of the guide rails; and the locking latch may be movable along the guide rails between the first and second latch positions.

In such embodiments of the invention the guide rails may support the locking latch within the latch receptacle to ensure it remains correctly orientated within the latch receptacle. This may be particularly advantageous when the locking latch is in the first latch position, engaged with the locking gear, and lateral forces are being applied to the locking latch by the locking gear that might otherwise twist or dislodge the locking latch from the correct orientation.

In embodiments of the invention each of the guide rails and guide apertures may extend linearly.

In such embodiments of the invention, the locking latch may be movable only linearly between the first latch position (proximal to the rotation axis) and the second latch position (distal to the rotation axis).

In embodiments of the invention each of the guide rails may extend normally to the rotation axis.

In such embodiments of the invention, the locking latch may be movable only normally to the rotation axis and radially to the locking gear. Therefore, when the locking latch moves to the first latch position while the locking gear is rotating, there may be a reduced likelihood of the locking gear slipping past the locking latch and the engagement failing.

In embodiments of the invention the joint locking mechanism may further comprise a diametrically magnetized magnet coupled to the locking gear and a hall-effect sensor coupled to the latch receptacle, the diametrically magnetized magnet and hall-effect sensor positioned relative to one another such that the hall-effect sensor is able to sense a magnetic field of the diametrically magnetized magnet.

In such embodiments of the invention the rotational orientation of the locking gear relative to the latch receptacle may be determined via the sensing of the diametrically magnetized magnet's magnetic field by the hall-effect sensor.

In embodiments of the invention the joint locking mechanism may further comprise a capstan rotatable between a first capstan position and a second capstan position; and a tendon comprising a first end and a second end, the first end attachable to the capstan and the second end attachable to the locking latch, wherein when the capstan is positioned at the first capstan position, the locking latch is positioned at the first latch position, and when the capstan is positioned at the second capstan position, the locking latch is positioned at the second latch position.

In such embodiments of the invention the locking latch may be tendon driven. When the capstan rotates from the first capstan position to the second capstan position, the tendon may be wound around the capstan. Winding of the tendon around the capstan may cause tension in the tendon extending between the capstan and the locking latch to increase such that the tensile force is higher than the force exerted by the bias on the locking latch. Therefore, rotation of the capstan from the first capstan position to the second capstan position may cause the locking latch to move from the first latch position to the second latch position.

Conversely, when the capstan rotates from the second capstan position to the first capstan position the tendon may be unwound from the capstan, thereby reducing the tension in the tendon. Once the capstan is in the first capstan position, the tensile force may be lower than the force exerted by the bias on the locking latch, hence causing the causing the locking latch to move to the first position.

In embodiments of the invention the tendon may form part of a Bowden cable that also comprises a Bowden casing slidably encasing a portion of the tendon between its first end and second end.

In such embodiments of the invention the tendon may be directed between the capstan and the locking latch by the Bowden casing. Further, the Bowden casing may protect the tendon as it extends between the capstan and the locking latch. The Bowden cable may therefore facilitate the capstan being positioned distally to the locking latch while ensuring that the tendon is properly positioned and protected as it extends between the capstan and locking latch.

In embodiments of the invention the locking latch may comprise a tendon aperture extending through the locking latch, the tendon extending through the tendon aperture, and a ferrule attachable to the second end of the tendon such that the second end of the tendon is attached to the locking latch.

In such embodiments of the invention, the tendon aperture may extend through the locking latch in a direction that is one or more of: normal to the rotation axis, radial to the locking gear, parallel to the direction in which the locking receptacle extends and parallel to the direction in which the bias forces the locking latch. By virtue of the tendon extending through the tendon aperture, the locking latch may be urged to move in an optimal direction when pulled by the tendon.

According to a second aspect of the invention there is provided a lockable joint assembly comprising: a first joint locking mechanism according to the first aspect of the invention, a first body, and a second body attachable to the first body, wherein: the rotation axis of the first joint locking mechanism is a first rotation axis; the second body is rotatable relative to the first body about the first rotation axis; the locking latch of the first joint locking mechanism is movably couplable to the first body; and the locking gear of the first joint locking mechanism is rigidly couplable to the second body, such that, when the locking latch of the first joint locking mechanism is in the respective first latch position the second body is locked in position relative to the first body; and when the locking latch of the first joint locking mechanism is in the respective second latch position the second body is rotatable relative to the first body about the rotation axis.

By means of the invention the first body and second body may form a first rotatable joint which may be selectively allowed or prevented from rotating about the first rotation axis by an operator of the lockable joint assembly using the first joint locking mechanism. The first rotatable joint may provide the lockable joint assembly with a first degree of freedom.

In some embodiments of the invention the first rotatable joint may be a passive joint wherein the stiffness of the joint is constant. In such embodiments of the invention the first joint locking mechanism may provide an operator of the lockable joint assembly with a means for selectively preventing the second body from rotating relative to the first body where there would otherwise be no such means. However, in W other embodiments of the invention the first rotatable joint may be an active joint wherein the stiffness of the joint may be actively varied. In such embodiments of the invention the first joint locking mechanism may, for example, be used as a fail-safe means for preventing the second body from rotating relative to the first body.

A latch receptacle forming part of the first joint locking mechanism may be rigidly couplable to the first body such that it is fixedly positioned relative to the first rotation axis. The locking latch may therefore be movably coupled to the first body by being movably received within the latch receptacle.

In embodiments of the invention the lockable joint assembly may further comprise a second joint locking mechanism according to the first aspect of the invention; a third body attachable to the second body wherein: the rotation axis of the second joint locking mechanism is a second rotation axis; the third body is rotatable relative to the second body about the second rotation axis; the locking latch of the second joint locking mechanism is movably couplable to the second body; and the locking gear of the second joint locking mechanism is rigidly couplable to the third body, such that, when the locking latch of the second joint locking mechanism is in the respective first latch position the third body is locked in position relative to the second body; and when the locking latch of the second joint locking mechanism is in the respective second latch position the third body is rotatable relative to the second body about the rotation axis.

In such embodiments the second body and third body may form a second rotatable joint which may be selectively allowed or prevented from rotating about the second rotation axis by an operator of the lockable joint assembly using the second joint locking mechanism. The second rotatable joint may provide the lockable joint assembly with a second degree of freedom.

In some embodiments of the invention the second rotatable joint may be a passive joint wherein the stiffness of the joint is constant. In such embodiments of the invention the second joint locking mechanism may provide an operator of the lockable joint assembly with a means for selectively preventing the third body from rotating relative to the second body where there would otherwise be no such means. However, in other embodiments of the invention the second rotatable joint may be an active joint wherein the stiffness of the joint may be actively varied. In such embodiments of the invention the second joint locking mechanism may, for example, be used as a fail-safe means for preventing the third body from rotating relative to the second body. In embodiments of the invention the lockable joint assembly may further comprise a third joint locking mechanism according to the first aspect of the invention, a fourth body attachable to the third body wherein: the rotation axis of the third joint locking mechanism is a third rotation axis; the fourth body is rotatable relative to the third body about the third rotation axis; the locking latch of the third joint locking mechanism is movably couplable to the third body; and the locking gear of the third joint locking mechanism is rigidly couplable to the fourth body, such that, when the locking latch of the third joint locking mechanism is in the respective first latch position the fourth body is locked in position relative to the third body; and when the locking latch of the third joint locking mechanism is in the respective second latch position the fourth body is rotatable relative to the third body about the rotation axis.

In such embodiments the third body and fourth body may form a third rotatable joint which may be selectively allowed or prevented from rotating about the third rotation axis by an operator of the lockable joint assembly using the third joint locking mechanism. The third rotatable joint may provide the lockable joint assembly with a third degree of freedom.

Accordingly, a lockable joint assembly may be provided which three degrees of freedom. Each of the first, second and third rotation axes may extend normally to each of the other rotation axes.

In some embodiments of the invention the third rotatable joint may be a passive joint wherein the stiffness of the joint is constant. In such embodiments of the invention the third joint locking mechanism may provide an operator of the lockable joint assembly with a means for selectively preventing the fourth body from rotating relative to the third body where there would otherwise be no such means. However, in other embodiments of the invention the third rotatable joint may be an active joint wherein the stiffness of the joint may be actively varied. In such embodiments of the invention the third joint locking mechanism may, for example, be used as a fail-safe means for preventing the fourth body from rotating relative to the third body.

According to a third aspect of the invention there is provided a controller arm comprising a lockable joint assembly according to the second aspect of the invention and a motor, wherein: the or each joint locking mechanism is a joint locking mechanism according to embodiments of the first aspect of the invention comprising a capstan and a tendon; the capstan of the or each joint locking mechanism is rotatably drivable by the motor.

In such embodiments of the invention the controller arm may form part of a master controller. The controller arm may be manipulated by an operator in order to control a slave device. The angular position of each rotatable joint forming part of the lockable joint assembly may be determined from data recorded by a Hall-effect sensor forming part of each joint locking mechanism. This data may be transmitted to a slave device in order that the slave device replicates the movements of the controller arm caused by an operator.

In some embodiments of the invention, the lockable joint assembly may comprise more than one joint locking mechanism and, therefore, more than one capstan. However, all of the capstans may be driven by a single motor such that each capstan may be simultaneously rotated between its first capstan position and its second capstan position. Accordingly a single motor may be used to lock and unlock each rotatable joint of the lockable joint assembly.

However, other embodiments of the invention may comprise more than one motor. For example, embodiments of the invention may comprise a different motor to drive each capstan such that each joint locking mechanism may be operated independently.

In embodiments of the invention the controller arm may further comprise a handle and a finger gripper, each coupled to the lockable joint assembly.

In such embodiments of the invention an operator may hold the handle and manipulate the orientation of the handle to cause rotation of each rotatable joint forming part of the lockable joint assembly. The operator's manipulation of the handle may be replicated by a slave device.

In embodiments of the invention the controller arm may further comprise an active joint assembly and a base, wherein: a proximal end of the active joint assembly, the motor and the capstan of the or each joint locking mechanism are each couplable to the base; a distal end of the active joint assembly is couplable to the lockable joint assembly.

In such embodiments of the invention the base may be stationary in use and the handle may freely be manipulated by an operator in use. The lockable joint assembly may provide the handle with three degrees of freedom relative to the base. For example, the lockable joint assembly may provide three rotational degrees of freedom in order that the operator may control the orientation of handle and, in turn, the slave device.

The active joint assembly may also provide the handle with three degrees of freedom relative to the base. However, the active joint assembly may provide translational degrees of freedom rather than rotational degrees of freedom. Accordingly, the handle may be provided with six degrees of freedom relative to the base. The controller arm may therefore allow an operator to control a slave device with six degrees of freedom.

The motor for actuating the capstans of the joint locking mechanism may be coupled to the base so that it remains stationary in use, rather than being mounted to a movable portion of the controller arm. This avoids the movable parts of the controller arm being bulky or heavy due to the motor being mounted to them. However, the tendons may allow the actuations of the motor to be transferred from the capstans to the locking latches of the joint locking mechanisms despite the active joint assembly being positioned between the base and the lockable joint assembly.

In embodiments of the invention the controller arm may comprise other combinations of joint assemblies. For example, the controller arm may comprise any combination of: one or more lockable joint assemblies; zero, one or more active joint assemblies; and zero, one or more passive joint assemblies.

According to a fourth aspect of the invention a master controller comprising a plurality of controller arms according to the third aspect of the invention.

By means of the invention a master controller may be provided with any suitable number of controller arms. For example, in some embodiments of the invention the master controller may comprise two controller arms, each of which may be controlled by one of the operator's hands. The operator may therefore provide controls to a slave device with both hands simultaneously. However, in other embodiments of the invention there may be more than two controller arms. This may allow an operator to swap between controller arms that provide different functionality with respect to a single slave device. Alternatively, different controller arms may control different slave devices. A master controller may also be provided that facilitates a team of operators providing controls to one or more slave devices simultaneously.

The invention will now be described by way of example only with reference to the accompanying drawings in which:

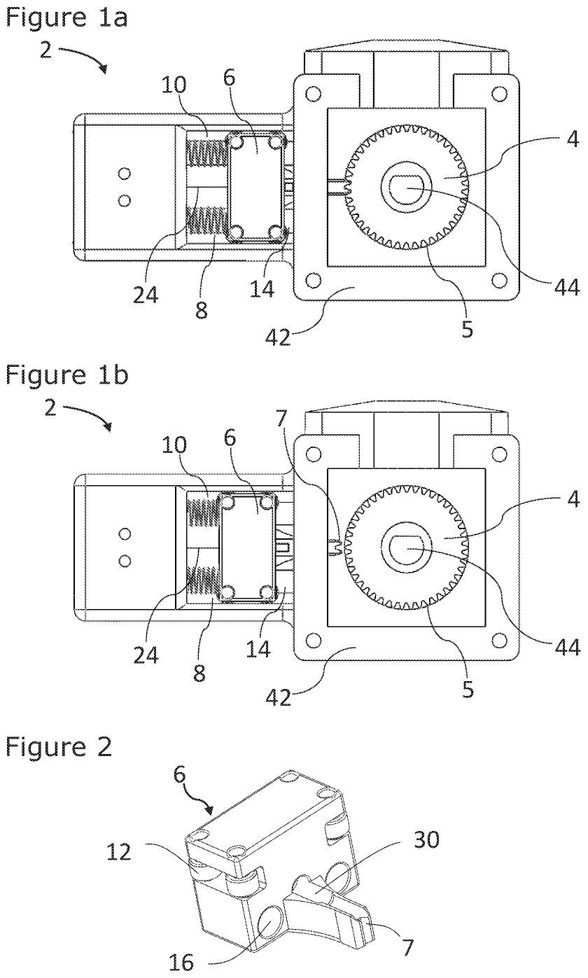

FIG. 1a is a schematic representation of a joint locking mechanism according to the first aspect of the invention, showing a locking latch in a first latch position;

FIG. 1b is a schematic representation of the joint locking mechanism shown in FIG. 1a with the locking latch in a second latch position;

FIG. 2 is a schematic representation of a locking latch forming part of the joint locking mechanism shown in FIG. 1a.

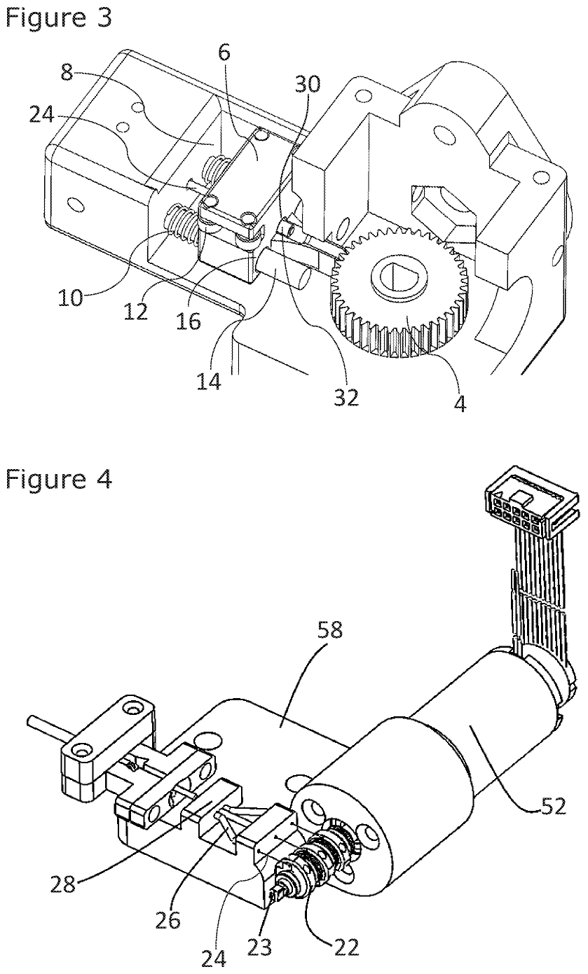

FIG. 3 is a schematic representation of the joint locking mechanism shown in FIG. 1a with additional features revealed.

FIG. 4 is a schematic representation of a plurality of capstans and tendons, each capstan and tendon forming part of a joint locking mechanism such as that shown in FIG. 1.

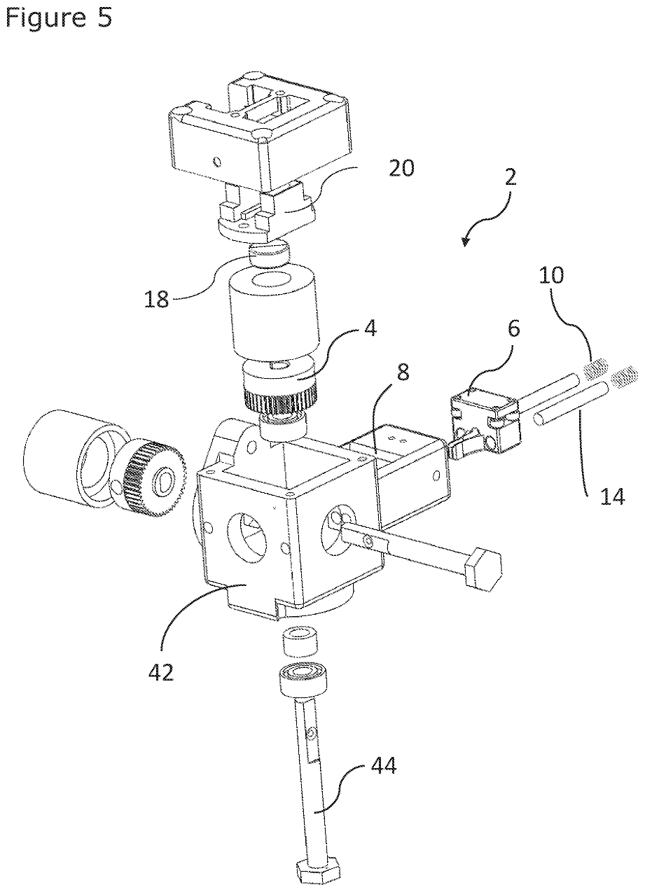

FIG. 5 is an exploded view of the joint locking mechanism shown in FIG. 1.

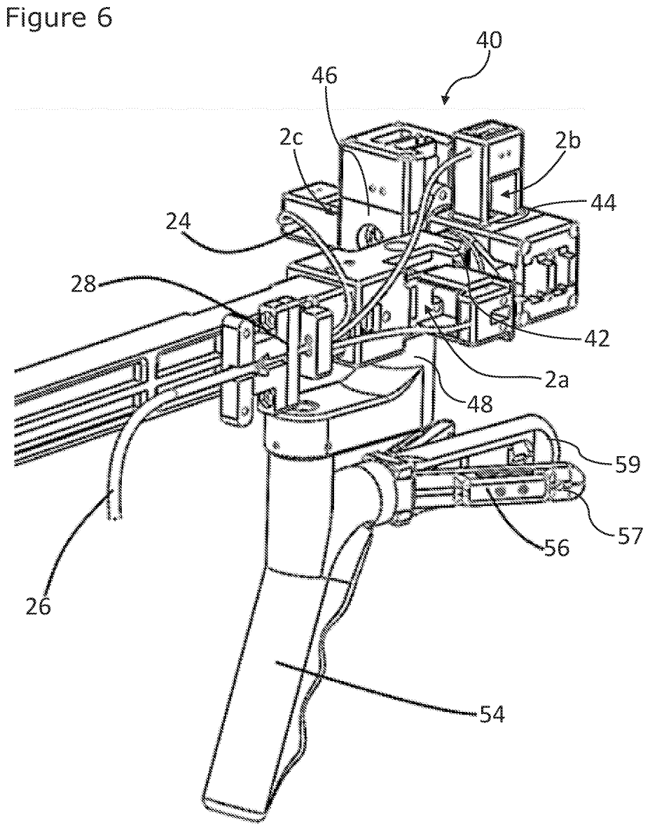

FIG. 6 is a schematic representation of a lockable joint assembly according to the second aspect of the invention.

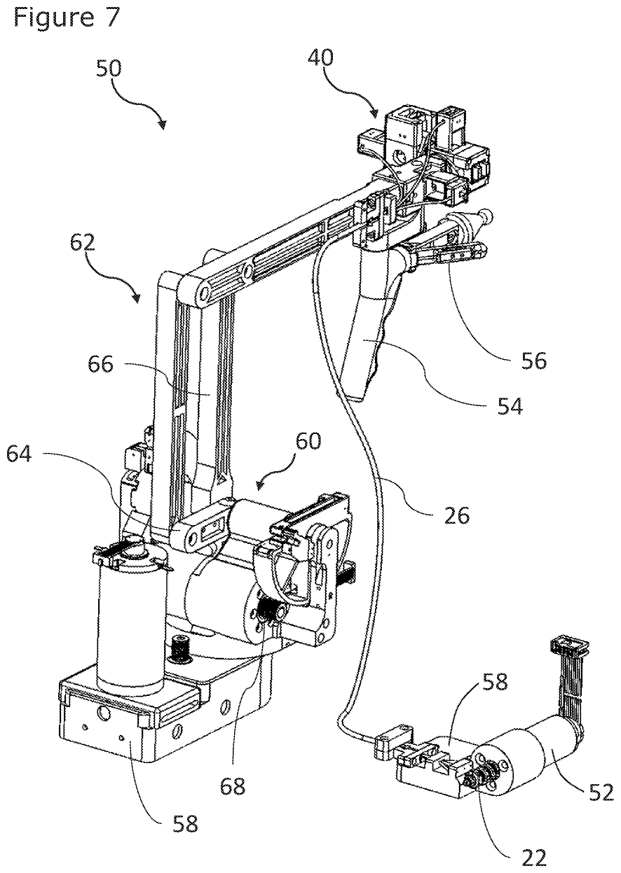

FIG. 7 is a schematic representation of a controller arm according to the third aspect of the invention.

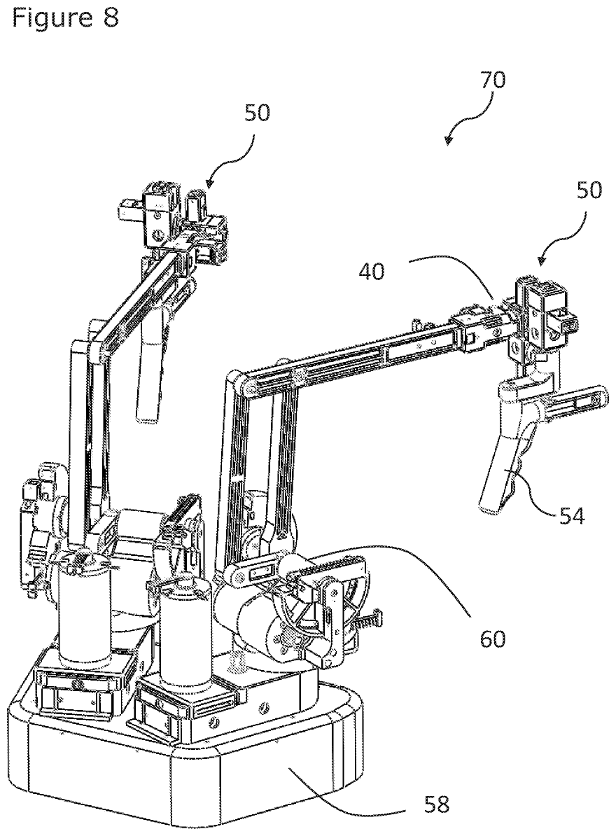

FIG. 8 is a schematic representation of a master controller according to the fourth aspect of the invention.

Referring initially to FIGS. 1a and 1b, a joint locking mechanism according to an embodiment of the invention is designated generally by the reference numeral 2. The joint locking mechanism 2 comprises a locking gear 4 rotatable about a rotation axis which extends centrally through the locking gear 4, and a locking latch 6 removably engageable with the locking gear 4 and movable relative to the rotation axis between a first latch position (as shown in FIG. 1a) and a second latch position (as shown in FIG. 1b). The locking gear 4 comprises a plurality of engagement teeth 5 extending radially from the circumference of the locking gear 4 and the locking latch 6 comprises an engagement portion 7 engageable with the engagement teeth 5.

When the locking latch 6 is in the first latch position, the engagement portion 7 is positioned to interlock with the engagement teeth 5 such that the locking latch 6 is engaged with the locking gear 4 and the locking gear 4 is locked in position relative to the rotation axis. Conversely, when the locking latch 6 is in the second latch position the locking latch 6 is spaced apart from the locking gear 4 and, in particular, the engagement portion 7 is spaced apart from the engagement teeth 5 such that the locking gear 4 is rotatable about the rotation axis.

The joint locking mechanism 2 further comprises a latch receptacle 8 fixedly positionable relative to the rotation axis wherein the locking latch 6 is movably receivable within the latch receptacle 8. The latch receptacle 8 extends linearly along a latch axis which extends normally to the rotation axis.

The latch receptacle 8 comprises two guide rails 14 and the joint locking mechanism 2 further comprises a tendon 24 attached to the locking latch 6. (However, in other embodiments of the invention the latch receptacle may comprise any suitable number of guide rails.)

The joint locking mechanism 2 also comprises a bias 10 couplable to the locking latch 6 and the latch receptacle 8 such that it extends between the latch receptacle 8 and the locking latch 6. In this embodiment of the invention, the bias 10 is a pair of springs that extend along the guide rails 14. The bias 10 biases the locking latch 6 towards the first latch position. Therefore, in order for the locking latch 6 to move to the second latch position against the bias 10, the tensile force in the tendon 24 must be increased such that it is higher than the biasing force acting in the opposite direction. For example, in this embodiment of the invention, the tensile force must be large enough to cause the springs to compress in order that the locking latch 6 may move from the first latch position to the second latch position. Conversely, in order to move the locking latch 6 from the second latch position to the first latch position the tensile force in the tendon 24 must be reduced such that the biasing force is higher and causes the locking latch 6 to move against the tensile force. In other words, the tensile force must be reduced so that the springs may expand and push the locking latch 6 to the first latch position.

Referring now to FIGS. 2 and 3, the locking latch 6 comprises a plurality of rollers 12. Each roller 12 is rotatable relative to the locking latch 6 and engageable with the latch receptacle 8. Movement of the locking latch 6 between the first and second positions may therefore be facilitated by the rollers 12 being able to roll along internal walls of the latch receptacle 8.

The locking latch 6 further comprises a plurality of guide apertures 16 wherein each guide aperture 16 is slidably engageable with a respective one of the guide rails 14. The locking latch 6 is therefore movable along the guide rails 14 between the first and second positions. Each of the guide rails 14 and guide apertures 16 extends linearly such that the locking latch 6 may move linearly between the first and second positions. Further, each of the guide rails 14 extends normally to the rotation axis (and parallel to the latch axis).

The locking latch 6 also comprises a tendon aperture 30 extending through the locking latch 6. The tendon 24 may extend through the tendon aperture 30 and a ferrule 32 may be attached to an end of the tendon 24 extending through the tendon aperture 30 such that the tendon 24 is attached to the locking latch 6. The tendon aperture 30 extends linearly through the locking latch 6 such that when the locking latch 6 is received within the latch receptacle 8, in use, the tendon aperture 30 extends parallel to the latch axis.

Accordingly, the shape and orientation of the latch receptacle 8, the guide rails 14 and guide apertures 16 and the tendon aperture 30 all urge the locking latch 6 to move both linearly and normally to the rotation axis. The locking latch 6 is therefore movable radially to the locking gear 4. This means that, when the locking latch 6 moves to the first latch position while the locking gear 4 is rotating, the engagement portion 7 may interlock with and abut against the moving engagement teeth 5 with reduced likelihood of the locking gear 4 slipping past the locking latch 6 and the engagement failing.

Referring now to FIG. 4, the joint locking mechanism 2 (shown in FIG. 1) may further comprise a capstan 22 rotatable between a first capstan position and a second capstan position. The tendon 24, which is also shown in FIG. 1 with an end attached to the locking latch 6, may be attached at its opposite end to the capstan 22. When the capstan 22 is positioned at the first capstan position the locking latch 6 is positioned at the first latch position and when the capstan 22 is positioned at the second capstan position the locking latch 6 is positioned at the second latch position.

In preparation for use, the capstan 22 may be rotatably mounted to a drive shaft 23 and rotated to the first capstan position wherein the tendon 24 is wound around the capstan such that slack is removed from the tendon 24 but the locking latch 6 is in the first latch position. The capstan 22 may then be locked to the driveshaft 23 such that it is fixedly mounted and ready for use.

In use, the driveshaft 23 and, in turn, the capstan 22 are rotatably driveable by a motor 52. The motor may rotate the driveshaft 23 and capstan 22 in order to move the capstan 22 to between the first and second capstan positions. As the capstan 22 rotates from the first capstan position to the second capstan position the tendon 24 may be increasingly wound around the capstan 22 to increase the tensile force in the tendon 22 and cause the locking latch 6 to move from the first latch position to the second latch position. Conversely, when the capstan 22 rotates from the second capstan position to the first capstan position the tendon 24 is unwound from the capstan 22, the tensile force in the tendon 24 is reduced and the locking latch 6 may return to the first latch position.

The tendon 24 forms part of a Bowden cable 26 that also comprises a Bowden casing slidably encasing a portion of the tendon that extends between the capstan 22 and the locking latch 6.

The motor 52 and capstan 22 may be mounted to a base 58.

Referring back to FIG. 1, the latch receptacle 8 is rigidly coupled to a first body 42 and the locking latch 6 is movably coupled to the first body 42 via the latch receptacle 8. Meanwhile, the locking gear 4 is rigidly coupled to a second body 44 that is rotatably attachable to the first body 42.

Referring now to FIG. 5, the joint locking mechanism 2 further comprises a diametrically magnetized magnet 18 coupled to the locking gear 4 and a hall-effect sensor 20 coupled to the latch receptacle 8 (via the first body 42). The diametrically magnetized magnet 18 and hall-effect sensor 20 may be positioned relative to one another such that the hall-effect sensor 20 is able to sense a magnetic field of the diametrically magnetized magnet 18. Accordingly, the rotational orientation of the locking gear 4 relative to the latch receptacle 8 may be determined via sensing of the diametrically magnetized magnet's magnetic field by the hall-effect sensor 20.

Referring now to FIG. 6, a lockable joint assembly 40 according to an embodiment of the second aspect of the invention comprises a first body 42, a second body 44 rotatably attached to the first body 42, a third body 46 rotatably attached to the second body 44 and a fourth body 48 rotatably attached to the third body 46. The lockable joint assembly also comprises a first joint locking mechanism 2a, a second joint locking mechanism 2b and a third joint locking mechanism 2c, each of which is equivalent to the joint locking mechanism 2 shown in FIGS. 1 to 4. The rotation axis of the first joint locking mechanism 2a is a first rotation axis, the rotation axis of the second joint locking mechanism 2b is a second rotation axis perpendicular to the first rotation axis and the rotation axis of the third joint locking mechanism 2c is a third rotation axis perpendicular to both the first and second rotation axes.

The locking latch, latch receptacle and hall-effect sensor of the first joint locking mechanism 2a are coupled to the first body 42 while the locking gear and diametrically magnetized magnet of the first joint locking mechanism 2a are attached to the second body 44. Accordingly, when the locking latch of the first joint locking mechanism 2a is in the respective first latch position the second body 44 is locked in position relative to the first body 42. Conversely, when the locking latch of the first joint locking mechanism 2a is in the respective second latch position the second body 44 is rotatable relative to the first body 42 about the first rotation axis.

The second joint locking mechanism 2b is configured similarly to the first joint locking mechanism 2a except that it is coupled to the second and third bodies 44, 46 rather than the first and second bodies 42, 44 respectively. Likewise, the third joint locking mechanism 2c is configured similarly to the first joint locking mechanism 2a except that it is coupled to the third and fourth bodies 46, 48 rather than the first and second bodies 42, 44 respectively.

A tendon 24 forming part of each joint locking mechanism 2a, 2b, 2c may extend from the respective joint locking mechanism 2a, 2b, 2c to a respective capstan 22 as shown in FIG. 4. Each capstan 22 may be mounted to the same driveshaft 23 and may be driven by the same motor 52.

The lockable joint assembly 40 may further comprise a tendon alignment channel 28 which aligns the tendons 24 together so that they may all be encased within a single Bowden cable 26 to protect the tendons as they extend towards the capstans 22. Briefly referring back to FIG. 4, another tendon alignment channel 28 may be provided to align the tendons 24 as they extend from the capstans 22.

The lockable joint assembly 40 may form part of a controller arm 50 (shown in FIG. 6). The controller arm may also comprise a handle 54 and a finger gripper 56 coupled to the lockable joint assembly (via the fourth body 48).

Rotation of the first, second, third and fourth bodies 42, 44, 46, 48 relative to one another may facilitate rotation of the handle 54 about three axes, therefore allowing an operator to manipulate the handle 54 with three rotational degrees of freedom.

In this embodiment of the invention, the finger gripper 56 comprises two levers 57 positioned either side of a central prong 59. The levers 57 of the finger gripper 56 may be manipulated by an operator's thumb and forefinger while the operator holds the handle 54 with his or her remaining fingers. Each lever 57 may comprise a magnet and the central prong 59 may comprise a hall-effect sensor able to sense the magnetic field generated by each magnet such that the position of the levers 57 may be determined. The operator may therefore manipulate the levers 57 in order to control an aspect of a slave device.

Referring now to FIG. 7, a controller arm 50 according to an embodiment of the third aspect of the invention comprises the lockable joint assembly 40, handle 54 and finger gripper 56 shown in FIG. 6. The controller arm further comprises a motor 52 (also shown in FIG. 4) that may drive capstans 22 forming part of the joint locking mechanisms which in turn form part of the lockable joint assembly 40. Here the Bowden cable 26 comprising the tendons 24 is shown extending from the capstans 22 to the lockable joint assembly 40.

The controller arm also comprises base 58 and an active joint assembly 60 coupling the lockable joint assembly 40 to the base 58.

In this embodiment of the invention, the active joint assembly 60 comprises a four-bar linkage 62 which, in turn, comprises a short linkage 64 and a long linkage 66. The short linkage 64 and long linkage 66 are rotatable relative to one another as well as being rotatable relative to the base 58 and therefore provide the handle 54 with three translational degrees of freedom relative to the base 58. The active joint assembly 60 also comprises an active motor assembly 68 configured to actively vary the stiffness of each rotatable joint forming part of the active joint assembly 60 such that the active joint assembly holds its position when there is no manipulation caused by an operator.

Referring now to FIG. 8, a master controller 70 according to a fourth aspect of the invention comprises a pair of controller arms 50 shown in FIG. 6. An operator of the master controller 70 may hold a handle 54 in each hand in order to simultaneously manipulate each controller arm 50.

Motion data recorded by the master controller, by the hall-effect sensor in each joint locking mechanism (shown in FIG. 5) for example, may be converted to motor commands for a slave device (not shown) such that the slave device replicates the movements of the operator.

In use, when the operator is intending to control the slave device via manipulation of the master controller 70, the motor 52 may be caused to rotate the capstans 22 and tighten each of the tendons 24 such that the locking latch 6 of each joint locking mechanism 2a, 2b, 2c is moved to the second latch position and the first, second, third and fourth bodies are free to rotate relative to one another.

During use, it is possible that the operator may need to adjust the workspace of the master controller 70 relative to that of the slave device, for example to use the master controller 70 in a more comfortable position or to access a different portion of the slave device's workspace. In order to do this, the operator may clutch or disengage the master controller 70 from controlling the slave device. For example, the master controller may comprise a clutch pedal (or any other suitable clutch control mechanism), which when actuated prevents new motion commands being transmitted to the slave device based on movements of the controller arms 50.

However, to ensure that the orientation of the master controller handles 54 do not become misaligned with the orientation of the slave device during clutching, actuation of the clutch pedal may cause the motor 52 to rotate the capstans 22 to their respective first capstan positions, releasing the respective tendons 24 and causing the respective locking gears 4 to be locked in position.

Accordingly, the lockable joint assembly 40 may hold its position during the clutching of the master controller 70 from the slave device while the operator realigns the active joint assembly 60 through its translational degrees of freedom to a desired position. This means that the orientation of the handles 54 may remain aligned with the last orientation of the slave device set prior to clutching so that, when the operator resumes control of the slave device, any issues of perceiving misalignment between the orientation of the master controller 70 and slave device are obviated.

Once the clutching process is complete, release of the clutch pedal may cause the motor 52 to rotate the capstans 22 to their respective second capstan positions such that the lockable joint assembly is unlocked, and the operator may resume manipulating the handles 54 through three rotational degrees of freedom as well as three translational degrees of freedom.

Preferences and options for a given aspect, feature or parameter of the invention should, unless the context indicates otherwise, be regarded as having been disclosed in combination with any and all preferences and options for all other aspects, features and parameters of the invention. For example, the locking latch may comprise any suitable number of guide apertures, any suitable number of rollers, a tendon aperture or any combination of these features. It is to be understood that the locking latch 6 shown in FIG. 2 is merely one example of how these features may be combined.

1. A joint locking mechanism comprising:
a locking gear rotatable about a rotation axis;
a locking latch removably engageable with the locking gear and movable relative to the rotation axis between a first latch position and a second latch position; and
a bias couplable to the locking latch such that the locking latch is biased towards the first latch position,
wherein in the first latch position the locking latch is engaged with the locking gear such that the locking gear is locked in position relative to the rotation axis and in the second latch position the locking latch is spaced apart from the locking gear such that the locking gear is rotatable about the rotation axis.

2. A joint locking mechanism according to claim 1 wherein the locking latch is movable between the first latch position and the second latch position in a direction normal to the rotation axis.

3. A joint locking mechanism according to claim 1, further comprising a latch receptacle fixedly positionable relative to the rotation axis wherein the locking latch is movably receivable within the latch receptacle and the bias is couplable to the latch receptacle such that it extends between the latch receptacle and the locking latch.

4. A joint locking mechanism according to claim 3 wherein the locking latch comprises a plurality of roller, each roller rotatable relative to the locking latch and engageable with the latch receptacle.

5. A joint locking mechanism according to claim 4 wherein the latch receptacle extends linearly along a latch axis.

6. A joint locking mechanism according to claim 5 wherein the latch axis extends normally to the rotation

7. A joint locking mechanism according to claim 3 wherein:
the latch receptacle comprises a plurality of guide rails;
the locking latch comprises a plurality of guide apertures, each guide aperture slidably engageable with a respective one of the guide rails; and
the locking latch is movable along the guide rails between the first and second positions.

8. A joint locking mechanism according to claim 7 wherein each of the guide rails and guide apertures extends linearly.

9. A joint locking mechanism according to claim 8 wherein each of the guide rails extends normally to the rotation axis.

10. A joint locking mechanism according to claim 3 further comprising a diametrically magnetized magnet coupled to the locking gear and a hall-effect sensor coupled to the latch receptacle, the diametrically magnetized magnet and hall-effect sensor positioned relative to one another such that the hall-effect sensor is able to sense a magnetic field of the diametrically magnetized magnet.

11. A joint locking mechanism according to claim 1 further comprising:
a capstan rotatable between a first capstan position and a second capstan position; and
a tendon comprising a first end and a second end, the first end attachable to the capstan and the second end attachable to the locking latch, wherein when the capstan is positioned at the first capstan position the locking latch is positioned at the first latch position and when the capstan is positioned at the second capstan position the locking latch is positioned at the second latch position.

12. A joint locking mechanism according to claim 11 wherein the tendon forms part of a Bowden cable that also comprises a Bowden casing slidably encasing a portion of the tendon between its first end and second end.

13. A joint locking mechanism according to claim 11 wherein the locking latch comprises a tendon aperture extending through the locking latch, the tendon extending through the tendon aperture, and a ferrule attachable to the second end of the tendon such that the second end of the tendon attached to the locking latch.

14. A lockable joint assembly comprising: a first joint locking mechanism according to claim 1, a first body, and a second body attachable to the first body, wherein:
the rotation axis of the first joint locking mechanism is a first rotation axis;
the second body is rotatable relative to the first body about the first rotation axis;
the locking latch of the first joint locking mechanism is movably couplable to the first body; and
the locking gear of the first joint locking mechanism is rigidly couplable to the second body,
such that, when the locking latch of the first joint locking mechanism is in the respective first latch position the second body is locked in position relative to the first body; and
when the locking latch of the first joint locking mechanism is in the respective second latch position the second body is rotatable relative to the first body about the rotation axis.

15. A lockable joint assembly according to claim 14 further comprising a second joint locking mechanism comprising a locking gear rotatable about a rotation axis;
a locking latch removably engageable with the locking gear and movable relative to the rotation axis between a first latch position and a second latch position; and
a bias couplable to the locking latch such that the locking latch is biased towards the first latch position,
wherein in the first latch position the locking latch is engaged with the locking gear such that the locking gear is locked in position relative to the rotation axis and in the second latch position the locking latch is spaced apart from the locking gear such that the locking gear is rotatable about the rotation axis; a third body attachable to the second body wherein:
the rotation axis of the second joint locking mechanism is a second rotation axis;
the third body is rotatable relative to the second body about the second rotation axis;
the locking latch of the second joint locking mechanism s movably couplable to the second body; and
the locking gear of the second joint locking mechanism is rigidly couplable to the third body,
such that, when the locking latch of the second joint locking mechanism in the respective first latch position the third body is locked in position relative to the second body; and
when the locking latch of the second joint locking mechanism is in the respective second latch position the third body is rotatable relative to the second body about the rotation axis.

16. A lockable joint assembly according to claim 15 further comprising a third joint locking mechanism comprising a locking gear rotatable about a rotation axis;
a locking latch removably engageable with the locking gear and movable relative to the rotation axis between a first latch position and a second latch position; and
a bias couplable to the locking latch such that the locking latch is biased towards the first latch position,
wherein in the first latch position the locking latch is engaged with the locking gear such that the locking gear is locked in position relative to the rotation axis and in the second latch position the locking latch is spaced apart from the locking gear such that the locking gear is rotatable about the rotation axis; a fourth body attachable to the third body wherein,
the rotation axis of the third joint locking mechanism is a third rotation axis;
the fourth body is rotatable relative to the third body about the third rotation axis;
the locking latch of the third joint locking mechanism is movably couplable to the third body; and
the locking gear of the third joint locking mechanisms rigidly couplable to the fourth body,
such that, when the locking latch of the third joint locking mechanism is in the respect first latch position the fourth body is locked in position relative to the third body; and
when the locking latch of the third joint locking mechanism is in the respective second latch position the fourth body is rotatable relative to the third body about the rotation axis.

17. A controller arm comprising a lockable joint assembly according to claim 14 and a motor, wherein:
the or each joint locking mechanism is a joint locking mechanism according to claim 11;
the capstan of the or each joint locking mechanism is rotatably drivable by the motor.

18. A controller arm according to claim 17 further comprising a handle and a finger gripper, each coupled to the lockable joint assembly.

19. A controller arm according to claim 18 further comprising an active joint assembly and a base, wherein:
a proximal end of the active joint assembly, the motor and the capstan of the or each joint locking mechanism are each couplable to the base;
a distal end of the active joint assembly is couplable to the lockable joint assembly.

20. A faster controller comprising a plurality of controller arms according to claim 18.
